(19) United States
(12) Patent Application Publication
(10) Pub. No.: US 2007/0254035 A1
(43) Pub. Date: Nov. 1, 2007

(54) Preparing Active Polymer Extrudates

(76) Inventors: Jianyuan Hao, Nottingham (GB); Martin James Whitaker, Nottingham (GB); Kevin Morris Shakesheff, Nottingham (GB); Steven Melvyn Howdle, Nottingham (GB)

Correspondence Address: WILMER CUTLER PICKERING HALE AND DORR LLP, 60 STATE STREET, BOSTON, MA, 02109, US

(21) Appl. No.: 10/576,909

(22) PCT Filed: Oct. 22, 2004
(86) PCT No.: PCT/GB04/04470

§ 371 Date: Jun. 29, 2007

(30) Foreign Application Priority Data
Oct. 23, 2003 (GB) 0324720.2
Feb. 14, 2004 (GB) 0403361.9

(51) Int. Cl. C08J 7/12; A61K 9/14; B01J 3/00; C08J 3/205

(52) U.S. Cl. 424/486; 264/171.1; 264/171.26; 264/172.11; 425/66; 514/772.3

(57) ABSTRACT

Process for preparing active polymer extrudate comprising polymer matrix and guest matter, the process comprising contacting a polymer substrate and guest matter with a plasticising fluid under dense phase, sub critical or supercritical plasticising conditions of elevated temperature and/or pressure to plasticise the polymer substrate and incorporate guest matter and extruding polymer substrate incorporating guest matter under dense phase, sub critical or supercritical conditions via an extrusion orifice into a collection zone or a mould with simultaneous or subsequent release of pressure, whereby extrudate is obtained comprising a solid admixture of polymer matrix and guest matter in form conferred by the orifice or the mould; a novel extrudate; composition thereof and apparatus for the preparation thereof, and use thereof in fibre processing techniques, medical applications such as in delivery of drugs and other agents such as imaging and diagnostic agents, tissue engineering, and as medical devices or aids such as delivery devices or aids for drugs, imaging and diagnostic agents, as tissue engineering devices or aids such as sutures, and the like; as an anti-microbial for example having bacteria-static or -cidal activity; as a natural or synthetic barrier capable of immobilising e.g. naturally occurring or artificially introduced poisons or toxins by e.g. absorption, interaction or reaction; in agrochemical or crop protection applications; in the processing of thermally labile fibres for use in dying, textiles, electronics etc below the polymer Tg, Tm or viscosity; in incorporation of dyes and other thermally labile materials into polymers that cannot be formed by traditional processes e.g. melt extrusion and the like; or in incorporation of surfactants into fibres to control polymer properties.

The present invention relates to a process for the preparation of active polymer extrudates by plasticising a polymer and extruding through an orifice, novel extrudates, compositions thereof use thereof in or in association with animals or humans, cultivated or uncultivated matter, and apparatus for the preparation thereof. More particularly the present invention relates to a process for the preparation of active polymer extrudates incorporating guest matter such as dye, drug, protein, metal or other molecules by plasticising polymer and guest matter and extruding through an orifice, active polymer extrudates as solid admixtures with guest matter in the form of sheets, films, tubes, cylinders, ribbons, fibrils, fibroids, fibres, non-woven materials and the like, compositions thereof, apparatus for the preparation thereof; and use thereof in fibre processing techniques, medical applications, as a natural or synthetic barrier, in agrochemical or crop protection applications, in the processing of thermally labile fibres for use in dyeing, textiles, electronics etc below the polymer Tg, Tm or viscosity in incorporation of dyes and other thermally labile guest matter into polymers that cannot be formed by traditional processes.

It is known that supercritical fluid (SCF) mixing can be used to incorporate inorganic materials and, more recently, biologically active species, into polymer hosts, the latter without loss of activity. For example, CO₂-induced plasticisation has been exploited to lower the viscosity of biodegradable polymers such as poly(DL-lactide) (PLA), polylactide-co-polygycolide (PLGA) and poly(ε-caprolactone), to such an extent that bio-active guest could be mixed into the polymer at pressures close to ambient such as 35° C., 200 bar (Howdle at al, Chem. Commun. 2001,190). Polymers incorporating such bio-active guest have been prepared as foamed monoliths and as powders, which are suitable for a wide range of medical applications including tissue engineering, drug release and the like.

In the field of drug release it is known to prepare biodegradable polymer fibres incorporating bio-active materials, which degrade to release those materials. Fibres may be injected or inserted subcutaneously or intramuscularly and have the advantage that a single fibre can stay in place, in contrast to several particles which move around. An advantage of fibre release materials over conventional powder or particle release materials is the ability to precisely locate fibres for desired drug release or implantation.

Traditional methods for incorporating guest matter into polymer fibres include impregnation of existing fibres with a solution of the guest matter. However this is inefficient requiring two stages of processing. Moreover the need to use solvents to impregnate leads to problems with residues, deactivation of bio-active materials and the like.

Traditionally polymer fibres are formed by melt extrusion, whereby polymer beads are melted and the melt extruded through an orifice in manner to generate fibres. More recently processes have been developed for the production of polymer fibres by saturation of polymer with supercritical CO₂ and spinning and extruding to form fibres, in which processing is conducted at temperatures below the melt temperature or below the Tg of the polymer. However these processes are typically conducted at relatively high temperatures typically in excess of 200° C. for certain polymers/polymer types. Accordingly such processes would not be suitable for fibre formation with temperature labile polymers or with temperature sensitive bio-active guest matter.

Specifically there are two types of polymer fibre manufacturing process using SCF: continuous and discontinuous. Both rely on the polymer being saturated with a fluid or gas under pressure to induce foaming and then quenching of system by either releasing pressure or increasing the temperature. The foaming takes place as polymer gas mixture is extruded in the continuous process or foaming is caused by pressure release or immersion in a heating bath prior to extrusion, in the discontinuous process. Both of these fibre producing processes suffer from the disadvantage that the saturation and foaming conditions are coupled whereby there is a low amount of control of fibre processing.

We have now found that a process can be provided for preparing fibre form polymer extrudates incorporating guest matter, by means of SCF processing in a single stage, and with independent control of SCF saturation and of fluid plasticisation and of polymer foaming conditions. It is particularly advantageous and indeed particularly surprising that these techniques can be combined in a single stage process in view of the requirement for polymer of sufficient cohesion to form fibres, typically achieved by going to higher molecular weights and more viscous materials, contrasted with the requirement for polymer of sufficiently low viscosity to allow incorporation of guest matter. However our work has led to the finding that the process of the invention allows manipulation of process conditions, in the form of temperature and pressure, polymer properties in the form of molecular weight and inherent viscosity, hardware characteristics such as extrusion orifice dimensions and in particular SCF temperature and extrusion back-pressure, whereby it is possible to achieve guest matter incorporation and fibre formation in a single stage. The process has particular benefits in that it allows operation without solvent if desired, at moderate temperatures which allow activity of guest matter to be completely or predominantly retained, and with incorporation of guest matter in very low or intermediate or very high levels, as desired, with homogeneous distribution in extrudates of desired shape and size, and of desired properties such as porosity and the like.

In the broadest aspect in the invention there is therefore provided a process for preparing active polymer extrudate comprising polymer matrix and guest matter, the process comprising contacting a polymer substrate and guest matter with a plasticising fluid under dense phase, sub critical or supercritical plasticising conditions of elevated temperature and/or pressure to plasticise the polymer substrate and incorporate guest matter and extruding polymer substrate incorporating guest matter under dense phase, sub critical or supercritical conditions via an extrusion orifice into a collection zone or a mould with simultaneous or subsequent release of pressure, whereby extrudate is obtained comprising a solid admixture of polymer matrix and guest matter in form conferred by the orifice or the mould.

Suitably extrudate according to the invention comprises any extrudate which is shaped or is continuous or coherent in at least one dimension. Preferably extrudate according to the invention comprises extrudate in the form of sheets, films, tubes, cylinders, rods, ribbons, fibrils, filaments, fibroids, fibres, solid and hollow sections, mesh, woven or non-woven extrudates and the like. Extrudate therefore excludes non shaped particles and powders which are known in the art and have been obtained by blasting polymer from a high pressure plasticizing vessel to a low pressure collection zone.

Reference herein to extruding or extrusion is to the operation of producing rods, tubes, sheets, film, wire coating and various solid and hollow sections by forcing suitable material, in this instance polymer material through a die by means of a ram, or of equivalent applied pressure, for example using a screw-drive, forcing through the fine holes of a spinneret or a nozzle under elevated pressure. Extrusion may be into a collection chamber or zone, or into a mould for subsequent shaping of extruded material, sometimes known as extrusion moulding. Reference herein to extrudate is to the product obtained thereby, having a form or shape conferred by the die through which extrusion occurred, or by a mould into which extrusion occurred, also known as moulded extrudate.

Reference herein to a solid admixture is to polymer matrix incorporating guest matter typically in the form- of phase inclusions, uniform or non-uniform particulate dispersions and the like or any such desired morphology. For example the guest matter may be encapsulated by the polymer substrate in the form of a coating, or may be homogeneously distributed throughout the polymer substrate.

The process of the invention is particularly advantageous in that, contrary to expectation we have been able to prepare extrudates using polymers which are conducive to incorporating guest matter and yet which are sufficiently cohesive to form shaped extrudates. These objectives require different, and usually mutually exclusive polymer properties. Incorporating guest matter requires a low viscosity, whereas making shaped extrudates requires a relatively high molecular weight conferring cohesion. In the process of the invention mixing or combining of polymer and guest matter, and extrusion thereof, takes place simultaneously or sequentially in a single stage, and this is clearly different from much of the prior art. In a particular advantage of the invention the process is tuneable to preparing extrudates of different morphologies (i.e. of different diameter, of different porosity, different length) and to preserving activity, for example biological activity or the like, of guest matter.

Polymer substrate is suitably provided in any desired form which is convenient for introducing to the process and is typically provided as solid phase particles or granules or pellets as known as in the art of melt extrusion, but may alternatively be provided in the form of solid blocks or monoliths. Alternatively polymer may be provided in fluid form but this is less convenient and less advantageous.

A plasticising fluid employed in the process of the invention may be any dense phase, subcritical, supercritical or like fluid, preferably which is characterised by properties which are both gas like and liquid like. In particular the fluid density and solubility properties resemble those of liquids whilst the viscosity, surface tension and fluid diffusion rate in any medium resemble those of a gas, allowing gas like penetration of the polymer substrate. Accordingly a plasticising fluid is able to render the polymer substrate in a plastic state conducive to incorporation of guest matter and extrusion of polymer substrate and transformation into a desired shape or configuration, in this case extrudates.

It is known that plasticising fluids, and supercritical fluids in particular, cause a significant depression in the glass transition temperature (T_{g}) or melt temperature (Tₘ) or viscosity of many polymers which means that the polymer may be kept in the liquid state at relatively low temperatures. By lowering the temperature or pressure, or both, the amount of plasticising fluid absorbed by the polymer is decreased, thus T_{g}, Tm or viscosity begins to rise to the point where the T_{g}, Tm or viscosity for the polymer is higher than the plasticising temperature at which point the polymer shape and configuration becomes fixed as the polymer solidifies.

In a particular advantage of the invention, the process may therefore be conducted in the substantial absence of additional solvent. This can be very significant in the case that guest matter has a desired activity or property which may be modified or destroyed by phase transition or solution. In a particular advantage of the invention therefore the process is suitable for preparation of polymer extrudates incorporating solvent sensitive guest matter, which may be in unchanged chemical form and/or unchanged physical form.

The process may be conducted in any suitable manner and is preferably conducted in an apparatus comprising a pressure chamber adapted for temperature and pressure elevation and which may comprise means for mixing the contents. The pressure chamber includes means for extruding contents via an orifice as hereinbefore defined into a collection zone or mould which may be at lower or higher pressure than the plasticising pressure. The apparatus comprises means for introduction of reactants and components whilst the pressure chamber is pressurised, as commonly known in the art, for maintaining a desired pressure during extrusion. Suitably an extrusion orifice comprises a die of desired shape, dimensions and length and may comprise a single or plural orifices for producing one or a plurality of extrudates or a composite extrudate. A collection zone may be open or closed such as a collection plate, tray or chamber. A mould may be any open or closed mould for conferring shape.

In a further advantage the process of the invention achieves highly satisfactory removal of residual monomers and the like, which may be present in the supplied polymer or bioactive. This is of particular advantage for toxicity purposes, whereby release of monomers and the like into the human or animal body is highly undesirable and is to be avoided.

It may nevertheless be desirable in certain instances to conduct the process in part or in whole in the presence of additional solvent, in the case that guest matter is solvent insensitive, for example to facilitate dispersion or to have a specific effect on extrudate morphology, e.g. on porosity. In such case a solvent may comprise any suitable solvent known in the art and may comprise a solvent in which the polymer substrate and/or guest matter is soluble or insoluble.

The process may be operated at any suitable plasticising time required to induce plasticisation of polymer and incorporation of guest matter for example in the range 2 millisecond to 72 hours. Longer plasticisation times of the order of up to 72 hours, for example 10 minutes to 72 hours or 2 minutes to 24 hours or 5 minutes to 2 hours may be suitable in the case of stable or otherwise insensitive substrate or guest matter and in the case that extended periods are desired to incorporate guest matter. Alternatively shorter plasticising periods of the order of 2 milliseconds up to 10 minutes, preferably 20 milliseconds to 5 minutes, more preferably 1 second to 1 minute, for example 2 to 30 seconds or 2 to 15 seconds may be suitable in the case of unstable or sensitive polymer substrate or guest matter or in the case that a non-uniform dispersion of guest matter is suitable.

The fluid, polymer substrate and guest matter may be combined in any desired order, prior to or during application of plasticising conditions. The process may be operated with introduction of plasticising fluid under plasticising conditions prior to contacting with polymer and guest matter, in which case plasticising period may be as long as 5 hours, alternatively fluid may be brought gradually or rapidly to plasticising conditions in contact with one or both of polymer substrate and guest matter, as desired.

The process may comprise introducing polymer substrate as its precursor monomers or oligomers and reacting in situ to form cured polymer or may comprise introducing polymer substrate in desired chemical form.

The process may be operated with or without mixing as hereinbefore defined.

The process may be operated at any suitable temperature in the range minus 200° C. to plus 500° C. Typically plasticising fluids may be brought into plasticising state at temperatures of between 0° C. to plus 300° C. at standard plasticising pressures of 7 to 1000 bar.

Preferably the plasticising conditions comprise a desired temperature less than, equal to or greater than the fluids critical temperature (Tc) in the range −200° C. to +500° C., preferably −200° C. to 200° C.

Selection of plasticising temperature is determined in part by the plasticising fluid and the nature of the polymer substrate to be plastisiced. However in a particular advantage of the invention we have found that the process is conducive to operating at lower temperatures in the range minus 200° C. to plus 200° C., more preferably minus 200° C. to plus 140° C., more preferably minus 150° C. to plus 100° C. More preferably −100 to +100° C., more preferably −20° C. to +100° C., most preferably 3 to 75° C. For most fluids this will be in the range approximately 10 to 15° C., 15 to 25° C., 25 to 30° C., 30 to 35° C., 35 to 45° C. or 45 to 55° C., most preferably approximately 28 to 33° C. (CO₂). Other sub ranges may be envisaged and are within the scope of the invention. Preferably the lowest temperature is employed which is compatible with sufficient lowering of the polymer Tg, Tm or viscosity to achieve plasticisation and incorporation of guest matter. To operate at ambient temperature, the process of the invention may require compensation by increase in pressure.

In a preferred embodiment of the invention the process is operated at temperatures of less than or equal to 200° C., for example less than or equal to 140° C. and represents an entirely new departure in the field of SCF extrusion technology for the preparation of polymer fibre extrudates. Conventionally SCF extrusion operates at elevated temperatures in excess of the polymers glass transition temperature (Tg), melt temperature (Tm) or highly viscous state, typically in excess of 200° C. Although it is known that SCF processing allows reduction in temperature at which the polymer attains fluid state, nevertheless the requirements to form coherent fibres requires higher molecular weight or higher viscosity polymers and operation at higher temperatures has been common practice in the art.

Accordingly in a preferred embodiment of the invention there is provided a process for preparing active polymer extrudate comprising polymer matrix and guest matter, the process comprising contacting a polymer substrate and guest matter with a plasticising fluid under dense phase, sub critical or supercritical plasticising conditions of elevated temperature and/or pressure to plasticise the polymer substrate and incorporate guest matter and extruding polymer substrate incorporating guest matter under dense phase, sub critical or supercritical conditions via an extrusion orifice into a collection zone or a mould with simultaneous or subsequent release of pressure, whereby coherent extrudate is obtained comprising a solid admixture of polymer matrix and guest matter in form conferred by the orifice or the mould characterised in that the process is conducted at temperature of less than or equal to 200° C. and/or less than the Tg, Tm or non-viscous state of the polymer substrate.

Typically plasticising fluids may be brought into plasticising state at pressures of between 7 to 1000 bar at standard plasticising temperatures of between 0° C. to plus 300° C. Preferably the plasticising conditions comprises a desired pressure less than, equal to or greater than the plasticising fluids critical pressure (Pc) from in excess of 1 bar to 1000 bar, preferably 2 to 800 bar, more preferably 2 to 400 bar, more preferably 5 to 75 bar for example 15 to 73 bar or 75 to 380 bar for example 110 to 360 bar. For most fluids this will be in the range approximately 30 to 40 bar, 40 to 50 bar, 50 to 60 bar, 60 to 75 bar or 75 to 125 bar or 125 to 380 bar, and is most preferably approximately 34 to 75 bar for dense phase, sub or supercritical CO₂. Other sub ranges may be envisaged and are within the scope of this invention.

Suitably however plasticising conditions are selected as known in the art to achieve a desired degree of plasticisation. Typically a desired degree of plasticisation will achieve a viscosity or pseudo viscosity decrease which enables incorporation of guest matter and extrusion of polymer substrate to extrudate of desired shape and configuration. Importantly the viscosity or level of pseudo viscosity reduction affects the form of extrudates, but this is a complex interaction with other variables including polymer substrate molecular weight, orifice dimensions and length, pressure drop at orifice and the like.

Measurement of absolute viscosity or viscosity reduction is neither straightforward nor highly accurate and it is therefore convenient to determine a desired reduction by controlling processing conditions, substrate and extrusion parameters. Moreover the polymer substrate may be in solid form whereby it is not viscous, and the process comprises rendering in plasticised form initially, and imparting a desired viscosity reduction. A suitable viscosity is preferably in the range 1-1,000,000 centipoises, more preferably 500-500,000 centipoises, more preferably 1000-100,000 centipoises and may be determined optically by means of e.g. capillary rheometer. Viscosity reduction may be facilitated by incorporation of additional solvents as hereinbefore defined. Alternatively or additionally viscosity reduction may be facilitated by blending, mixing, agitation or the like of the polymer substrate. Blending may be by physical pumping or otherwise displacing polymer substrate. Agitation maybe by aeration or fluidising gas flow or the like. Blending may be conducted with or without physical mixing of guest matter into polymer substrate, and may be conducted prior to introduction of guest matter or in the presence of guest matter.

The process of the invention may be conducted with any desired polymer substrate as known in the art and is suitably conducted with polymer of molecular weight in the range 1 to 10,000 kDa. In the current practice of SCF processing of polymer substrates incorporating bio-active material it is known to remove SCF in situ by depressurising a processing chamber to obtain a monolith, or to remove the polymer-bio-active mixture from the pressure chamber by spraying under sub-critical conditions to obtain powder or particle polymer of a desired particle size. Such processes typically operate with polymer substrate having molecular weight of order 20 kDa. We have now surprisingly found that by selection of higher molecular weight the polymer in the range as hereinbefore defined and preferably in the range 1 to 1000 kDa, more preferably 1 to 500 kDa, more preferably 1 to 250 kDa, more preferably 1 to 200 kDa, for example 1 to 50 or 50 to 200 kDa, more preferably 1 to 150 kDa, for example 1 to 30 or 30 to 150 kDa polymer substrate is suited both to incorporation of guest matter and to formation of extrudates. The selection of molecular weight is nevertheless dependent on the nature of polymer substrate and the viscosity reducing conditions employed. Suitably a polymer substrate is selected for which the internal cohesive force between the molecules is strong enough to overcome the break up of material during the extrusion process. By this means, even at ambient temperatures and atnospheric pressures, the formation of poorly coherent extrudates, such as thin fibres, will be suppressed and high quality extrudates of desired dimension, such as thick threads, may be produced. Accordingly polymer having strong internal cohesive forces may be employed at lower molecular weight in the above range and polymer having weaker internal cohesive force may be employed at higher molecular weight in the above range.

It will be hereby seen that the molecular weight of polymer substrate affects the morphology of extrudates. Additional features affecting the morphology of extrudates include process conditions. It is a particularly advantageous feature of the invention that by selecting suitable substrate properties, processing conditions and the like, extrudate may be obtained in desired form. Processing conditions affecting morphology include orifice dimension and length, extrusion time, plasticising pressure, back pressure into which polymer substrate is extruded and the like.

The polymer substrate for processing according to the process of the invention may comprise one or more polymers, and may be of same or different phase, same or different properties for example forming same or different porosity and the like. Two or more polymers may be a mixture or blend of polymers or may be discrete. One or more may be porous and one or more non-porous or of different porosity.

Moreover the guest matter may comprise a single or plural guest entities. Plural guest entities may comprise guest matter of one type for one intended function together with guest matter of another type for a same or different intended function, for example one or more drugs and one or more excipients or the like.

Plural polymer types and guest matter types may be introduced as mixtures or as discrete components. For example two or more polymer types may be contacted with plasticizing fluid as discrete components and co-extruded to form a composite extrudate having two or more polymer layers or zones. At least one of the two or more polymer components comprises guest matter.

For example one polymer component may comprise guest matter and a second polymer component may be guest-free, and may be extruded as a sheath layer or a blank layer in a composite extrudate, for example for delayed release or pulsed release applications. Alternatively the two or more polymer components may each comprise same or different guest matter providing a composite having different properties in different zones or comprising or releasing two or more incompatible guests or releasing two or more guests in different release profiles or the like.

An extrudate or composite extrudate according to the invention may be of any form as hereinbefore defined, and is preferably of tube, cylinder, fibre, sheet or film or shaped or hollow form, for example may be a hollow or filled composite tube or cylinder, a coannular fibre or coated fibre, mono or multifilament where a multifilament may be woven or braided or spun as known in the textiles art, or the like, or a monolayer or laminar film or sheet.

The process of the invention may be operated with any suitable orifice shape and dimension. Orifice shape and dimension affects the form of extrudates, i.e. sheet form, tubes, cylinders, ribbons fibres, fibrils, fibroids and woven or non-woven extrudates as hereinbefore defined. Height and width or diameter of orifice is typically selected according to a desired height and width or diameter of extrudate. Nevertheless the process of the invention is particularly suitable to operation with orifice dimensions in the range 0.001 to 10 millimetres, more preferably 0.001 millimetres to 2 millimetres, more preferably 0.005 millimetres to 2 millimetres, more preferably 0.005 millimetres to 1 millimetre for example 0.01 to 2 millimetres or 0.05 to 1 millimetre. As is known in the art polymer may expand on exiting the extrusion orifice and may lead to extrudates of larger dimension, depending on pressure, porosity and the like. More importantly however we have found that the length of extrusion orifice is highly significant to the process of the invention. Specifically we have found that relatively longer orifices generated improved cohesion and were conducive to the formation of extrudates of greater stability, greater length and in particular when preparing fibre form extrudate, producing greater amounts thereof. Without being limited to this theory we believe that passing polymer substrate through a relatively longer orifice is conducive to polymer aligning along the extrusion direction, favouring the break up of extruded polymer along the axis direction, leading to elongate extrudate such as long fibres. Preferably therefore the orifice is of length in the range 0.1 millimetre to 1 metre, more preferably 0.2 to 200 millimetre, for example 0.5 to 10 millimetre or 0.1 metre to 1 metre. In general the longer the nozzle the longer the fibre produced.

The orifice maybe of continuous profile and dimensions along its length or otherwise. Suitably the orifice is of increasing dimension along its length, preferably increasing at a first angle with respect to the axis and optionally at a second angle in respect to the axis at the orifice outlet. Increasing angle may be in one or two dimensions, for example for a sheet extrudate angle of increase may be in height only or in height and width, and for a tube, cylinder, fibre or like extrudate, angle may be in two dimensions forming an orifice of conical profile. Preferably a conical orifice provides an angle (angle to the axis) in the range greater than 0 degrees to 89.9 degrees, more preferably 45 to 80 degrees, more preferably 50 to 65 degrees, for example 60 degrees. An orifice may be one of a plurality of orifices which may be independent or which may be adjacently or coaxially or concentrically aligned to form a plurality of simple extrudates or to form a composite extrudate as hereinbefore defined. An orifice may additionally or alternatively comprise a solid core or the like, whereby hollow extrudate is obtained for example an annular orifice may provide tubes, cylinders or the like.

In one embodiment of the invention the process is an extrusion moulding process and polymer—guest matter mix is caused to exit the extrusion orifice and to enter a collection chamber or zone comprising one or more moulds or tools. A mould or tool may comprise one or more shaped cavities to receive extrudate, for example a mould may comprise one or more channels of smaller diameter than the extrusion orifice, adapted to mould extrudate into fibre forms.

The process of the invention operates with an extrusion force applied to the plasticized polymer—guest matter mix, to drive the extrusion. Preferably extrusion force is applied by means of pressure differential (extrusion chamber to collection chamber) or physical force such as a rod or ram or screw as known in the art. The extrusion force determines in part the nature of the extrudates obtained. Preferably extrusion force is any suitable force which produces extrudates in accordance with the invention as hereinbefore defined. It should be appreciated that operation with a greater extrusion force than is suitable for the present invention would have the effect of blasting polymer through the orifice in manner to break up any cohesive forces, and extrudate as hereinbefore defined would not be obtained, rather particles or powder would be obtained. The present invention provides for the first time a means to prepare extrudates in the form of shaped polymer products comprising guest matter without the need to go to excessive temperatures of lower polymer Mw to lower the Tg, Tm or viscosity to the degree necessary to achieve incorporation of guest matter.

The process of the invention may be operated with continuous or intermittent extrusion of polymer substrate and guest matter. Extrusion time may be selected in combination with desired morphology of extrudate and length of orifice, amongst other factors. Length of spray will affect length and/or morphology of product. It may therefore be convenient to operate with a stable extrusion which confers a cooling effect at the orifice with benefits on morphology of extrudates. Alternatively the process may be operated with intermittent extrusion, for example of the order of seconds.

The process may be operated at any suitable pressure as hereinbefore defined. Plasticising pressure is very significant to the reduction in viscosity of polymer substrate and, in combination with the pressure into which polymer substrate is extruded, has a very significant effect on the morphology and nature of extrudate. A process for extruding relatively lower molecular weight polymer substrate is more pressure independent than is that for extruding a more viscous higher molecular weight polymer. Accordingly in the preferred range of operation of the present invention the plasticising pressure becomes a highly significant variable. Preferably therefore the process of the invention operates with plasticising pressure in excess of 120 bar in ranges as hereinbefore defined and this has been found to greatly decrease the viscosity of polymer substrate.

Extrusion is suitably conducted at critical or subcritical conditions in the case of supercritical fluid processing, sub-dense phase or otherwise ambient conditions.

The process of the invention may be operated with extrusion into an ambient atmosphere or into an atmosphere at elevated “back pressure”. We have found that this is a significant factor in determining morphology of extrudate. In practice the process may therefore be operated by maintaining elevated pressure, modifying elevated pressure or releasing pressure past the extrusion orifice or mould.

Back pressure applied in a collection zone strongly affects fibre morphology, whereby extruding into air at atmospheric pressure generates void free extrudates, and extruding into a back pressure of any inert gas such as nitrogen at elevated pressure generates porous extrudates. Preferably when the pressure in the pressure chamber is relatively low (i.e. less than 140 bar), a back-pressure must be maintained in the collection zone so that the polymer does not solidify too quickly, blocking the nozzle and preventing fibre formation.

In a particular advantage therefore the invention enables control of degree of porosity by varying the back pressure in a collection chamber. Preferably extrusion is into a collection zone at positive, ambient or negative pressure, which may be greater or less than the plasticising pressure and is preferably in the range 50 to 140 bar or in the range 1 to 50 bar.

Collection zone pressure may be greater or less than the plasticising pressure in the pressure chamber and is suitably in the range 50 to 140 bar, preferably 70 to 140 bar, more preferably 80 to 125 bar, for example 90 bar, or in the range 1 to 50 bar, preferably 1 to 30 bar, more preferably 5 to 15 bar, for example 10 bar. Typically the process operated at lower or atmospheric back pressures leads to fine extrudates with high polymer alignment, and operation at higher back pressures leads to thicker extrudates with lower polymer alignment. In addition ambient or low back pressure favours production of extrudates with a fine porous structure or non-porous structure whilst extrusion at elevated back pressures favours production of extrudates with a highly porous or large pore structure.

The process of the invention may be a batch or continuous process, as known in the art. Suitably the process is a batch process whereby substrates are introduced to a pressure chamber, the chamber sealed and plasticising conditions attained, followed by extrusion by continuous or intermittent evacuation of the pressure chamber. Alternatively the process is a continuous process whereby substrates are continually supplied to a pressure chamber which is maintained at desired plasticising pressure, throughout continuous or intermittent evacuation of chamber contents.

The process of the invention may be conducted in any suitable apparatus comprising a pressure chamber and an extrusion orifice as hereinbefore defined providing a plasticising zone and an extrusion zone. Suitably the process is conducted in any suitable extrusion apparatus comprising for example an extruder barrel having a rotating screw member mounted therein and having a drive motor to drive the rotating screw member. The apparatus may comprise a hopper or other reservoir for polymer to be introduced into the extruder barrel. The apparatus may comprise integral means for introducing guest matter together with polymer, or may comprise an additional hopper or a reservoir for introducing guest matter directly to the extruder barrel. The extruder barrel may comprise heating means, for example a plurality of barrel heaters mounted in manner to heat the barrel. The apparatus additionally comprises a pressure inlet for connection to a plasticising fluid reservoir for plasticising fluid pressurised to a selected pressure by pressurising means. The pressure inlet comprises means for supplying a metered amount at a controlled rate to the extruder barrel. One or more pressure inlets may be located at one or several locations along the barrel as desired, via a pressure manifold as known in the art if desired.

The apparatus may comprise mixing means as hereinbefore defined, such as a plurality of blades or the like, or an extrusion screw may provide suitable mixing. Apparatus known in the art include an additional nucleation stage which may be present or absent in the apparatus for use in the invention and is preferably absent. The apparatus of the invention comprises an extrusion orifice preferably comprising a die or nozzle of shape and dimensions as herein defined. Suitably the apparatus comprises means for controlling flow rates of polymer, guest matter and plasticising fluid. Means for controlling flow rate are known in the art and include the extrusion screw, displacement pumps, metering pumps and the like. In addition flow rate may be controlled by designing the inlet locations for polymer, guest matter and fluid.

The apparatus may comprise one or a plurality of extrusion orifices in the form of extrusion nozzles as herein defined. If the process is conducted in an apparatus comprising a nozzle of known length and width as extrusion orifice, and conducted with use of a viscous polymer/plasticising fluid blend, a pressure drop results with increased length and decreased width due to friction in the nozzle. Suitably the apparatus of the invention is determined with reference to the number of extrusion orifices and orifice dimensions. For example with use of a single extrusion orifice the diameter and length are suitably selected to provide an appreciable pressure drop greater than a desired minimum pressure drop. The pressure drop may be controlled by configuring the extrusion orifice geometry as known in the art. By selection of suitable extrusion orifice thereby determining pressure drop, the pressure control within the apparatus may be controlled and the plasticising pressure may be maintained. It may be useful to provide additional positive pressure means to compensate for pressure loss at the extrusion orifice during continuous or intermittent extrusion. In a preferred embodiment of the invention, the extrusion orifice is selected in order to combine the requirement for sufficient length to give continuous extrudate formation, and to provide desired pressure drop at the orifice.

The polymer may be selected from any known polymer which is suited for the intended application. Suitably polymer is selected from any amorphous (solid, semi solid or fluid eg liquid) or semi-crystalline or crystalline polymers. Polymer suitable for introduction into or association with the human or animal body or living matter in non-toxic manner may be selected from synthetic biodegradable polymers as disclosed in “Polymeric Biomaterials” ed. Severian Dumitriu, ISBN 0-8247-8969-5, Publ. Marcel Dekker, New York, USA, 1994, synthetic non-biodegradable polymers; and natural polymers. Preferably the polymer is selected from homopolymers, block and random copolymers, polymeric blends and composites of monomers which may be straight chain, (hyper) branched or cross-linked.

Polymers may include but are not limited to the following which are given as illustration only:

Polyesters including poly(lactic acid), poly(glycolic acid), copolymers of lactic and glycolic acid, copolymers of lactic and glycolic acid with poly(ethyleneglycol), poly caprolactones such as poly gamma-caprolactone and poly(e-caprolactone), poly(3-hydroxybutyrate), poly(p-dioxanone), polydioxepanone, poly(propylene fumarate) and poly alkylene oxalates

Poly (ortho esters) including Polyol/diketene acetals addition polymers as described by Heller in: ACS Symposium Series 567, 292-305, 1994;

Polyanhydrides including poly(sebacic anhydride) (PSA), poly(carboxybisbarboxyphenoxyphenoxyhexane) (PCPP), poly[bis(p-carboxyphenoxy)methane] (PCPM), copolymers of SA, CPP and CPM, as described by Tamada and Langer in Journal of Biomaterials Science—Polymer Edition, 3, 315-353, 1992 and by Domb in Chapter 8 of the Handbook of Biodegradable Polymers, ed. Domb A. J. and Wiseman R. M., Harwood Academic Publishers;

Poly(amino acids);

Poly(pseudo amino acids) including those described by James and Kohn in pages 389403 of Controlled Drug Delivery Challenges and Strategies, American Chemical Society, Washington D.C.;

Polyphosphazenes including derivatives of poly[(dichloro) phosphazene], poly[(organo)phosphazenes], polymers described by Schacht in Biotechnology and Bioengineering, 52, 102-108, 1996; and

Azo polymers

Including those described by Lloyd in International Journal of Pharmaceutics, 106, 255-260, 1994;

Vinyl polymers including polyethylene, poly(ethylene-co-vinyl acetate), polypropylene, poly(vinyl chloride), poly(vinyl acetate), poly(vinyl alcohol) and copolymers of vinyl alcohol and vinyl acetate, poly(acrylic acid) poly(methacrylic acid), polyacrylamides, polymethacrylamides, polyacrylates, Poly(ethylene glycol), Poly(dimethyl siloxane), Polyurethanes such as ester urethanes or epoxy, bis-maleimides, methacrylates such as methyl or glycidyl methacrylate, Polycarbonates such as tri-methylene carbonate, di-methylene tri-methylene carbonate, Polystyrene and derivatives;

carbohydrates, polypeptides and proteins including:

Sugars or saccharides and their processable derivatives, Starch, Cellulose and derivatives including ethylcellulose, methylcellulose, ethylhydroxyethylcellulose, sodium carboxymethylcellulose; Collagen; Gelatin; Dextran and derivatives; Alginates; Chitin; and Chitosan;

The polymer may comprise any additional polymeric components having performance enhancing or controlling effect, for example determining the degree and nature of cross-linking for improved permeability by bodily fluids or pharmaceutically effective agent, flexural properties, release properties or general mechanical properties.

The guest matter may comprise any material which it is desired to incorporate into a polymer for any desired application. Suitably guest matter comprises biofunctional or non-biofunctional material including but not limited to:
(1) (pharmaceutical) drugs and veterinary products;
(2) agrochemicals as pest and plant growth control agents;
(3) human and animal healthcare products;
(4) human and animal growth promoting, structural, or cosmetic products including products intended for growth or repair or modelling of the skeleton, organs, dental structure and the like;
(5) absorbent biofunctional materials for poisons, toxins and the like;
(6) functioning matter such as any nutrient dependent, biological matter which is characterised by replication, division, regeneration, growth, proliferation or the like;
(7) organic or inorganic materials for use in dyeing, constructing textiles, electronic materials and the like;
(8) SMART materials.
(9) In addition guest matter may include formulating agents which stabilise or enhance the functional material.

Preferably a biofunctional material is selected from any materials adapted to perform a function on a desired biolocus comprising or otherwise associated with living matter, as hereinbefore defined. A biofunctional material may be bioactive, bioinert, biocidal or the like. Preferably a biofunctional material is adapted to induce growth, strengthen, supplement or enhance a desired human, animal or living matter host structure, or combat or protect against threats to the host structure or to the human or animal body in general. The material may be selected from any inorganic or organic material which is optionally substantially insoluble in supercritical fluid, in either or both of its non critical and supercritical states.

Preferably guest matter is selected from biofunctional material including but not limited to medical and veterinary products such as drugs and medical agents such as imaging or diagnostic agents; agrochemicals as pest and plant growth control agents; human and animal health products; human and animal growth promoting, structural, or cosmetic products including products intended for growth or repair or modelling of the skeleton, organs, dental structure and the like; absorbent biofunctional materials for poisons, toxins and the like; functioning matter such as any nutrient dependent, biological matter which is characterised by replication, division, regeneration, growth, proliferation or the like; or comprises function enhancing components such as growth promoters and the like; or comprises organic or inorganic materials for use in dyeing, constructing textiles, electronic materials and the like

(1) Pharmaceuticals and veterinary products, i.e. drugs, may be defined as any pharmacologically active compounds that alter physiological processes with the aim of treating, preventing, curing, mitigating or diagnosing a disease.

Drugs may be composed of inorganic or organic molecules, peptides, proteins, enzymes, oligosaccharides, carbohydrates, nucleic acids and the like.

Drugs may include but not be limited to compounds acting to treat the following:

Infections such as antiviral drugs, antibacterial drugs, antifungal drugs, antiprotozoal drugs, anthelmintics, for example antibacterials include, but are not limited to, penicillins for example benzylpenicillin, cephalosporins for example ceftazidime, tetracyclins for example teterecycline, aminoglycosides for example gentamicin, macrolides for example erythromycin and the following: clindamycin, chloramphenicol, vancomycin, teicoplanin, colomycin, co-trimoxazole and trimethoprim;

Cardiovascular system such as positive inotropic drugs, diuretics, anti-arrhythmic drugs, beta-adrenoceptor blocking drugs, calcium channel blockers, sympathomimetics, anticoagulants, antiplatelet drugs, fibrinolytic drugs, lipid-lowering drugs;

Gastro-intestinal system agents such as antacids, antispasniodics, ulcer-healing, drugs, anti-diarrhoeal drugs, laxatives, central nervous system, hypnotics and anxiolytics;

Antipsychotic drugs such as chloropromazine hydrochloride and “atypical” anti-psychotic drugs such as amisulpride, clozapine, olanzapine, quetiapine, risperidone, sertindole, flupenthixol decanoate, haloperidol decanoate, pipothiazine palmitrate and zuclopenthixol decanoate; antidepressants, central nervous system stimulants, appetite suppressants, drugs used to treat nausea and vomiting, analgesics, antiepileptics, drugs used in parkinsonism, drugs used in substance dependence; Malignant disease and immunosuppresion agents such as cytotoxic drugs, immune response modulators, sex hormones and antagonists of malignant diseases;

Respiratory system agents such as bronchodilators, corticosteroids, cromoglycate and related therapy, antihistamines, respiratory stirnulants, pulmonary surfactants, systemic nasal decongestants;

Antitumorals such as BCNU or 1,3-bis (2-chloroethyl)-1-nitrosourea, daunorubicin, doxorubicin, epirubicin, idarubicin, 4-demethoxydaunorubicin 3′-desamine-3′-(3-cyano-4-morpholinyl)-doxorubicin, 4-demethoxydaunorubicin-3′-desamine-3′-(2-methoxy-4-morpholinyl)-doxorubicin, etoposide and teniposide;

Enzymes and hormones such as ribonuclease, lysozyme, and therapeutic proteins and enzymes listed in “Novel Therapeutic Proteins”, Klaus Dembowsky (Ed), Peter Stadler (Ed), Wiley-VCH Verlag GmbH, D-69469, Weinheim, Germany, 2001; LHRH and LHRH analogues, parathyroid hormone and analogues;

Steroideals for birth control and/or antitumoral action such as medroxyprogesterone acetate or megestrol acetate;

Musculoskeletal and joint diseases agents such as drugs used in rheumatic diseases, drugs used in neuromuscular disorders; and

Immunological products and vaccines.

Medical agents such as imaging or diagnostic agents may comprise any fluorescent or radioactive agents which are delivered to assist in imaging or diagnosis of the human or animal body, for example imaging or diagnostic agents intended for accumulation in body tissues or organs which allow the tissue or organ to be imaged, for diagnosing conditions such as cancer, lung disorders, liver and kidney disorders, bowel disorders and the like. Such agents are known in the respective arts.

(2) Agrochemicals and crop protection products may be defined as any pest or plant growth control agents, plant disease control agents, soil improvement agents and the like. For example pest growth control agents include insecticides, miticides, rodenticides, molluscicides, slugicides, vermicides (nematodes, anthelmintics), soil fumigants, pest repellants and attractants such as pheromones etc, chemical warfare agents, and biological control agents such as microorganisms, predators and natural products;

plant growth control agents include herbicides, weedicides, defoliants, dessicants, fruit drop and set controllers, rooting compounds, sprouting inhibitors, growth stimulants and retardants, moss and lichen controllers and plant genetic controllers or agents;

plant disease control agents include fungicides, viricides, timber preservatives and bactericides; and

soil improvement agents include fertilisers, trace metal additives, bacterial action control stimulants and soil consolidation agents.

(3) human and animal healthcare products may be defined as any of the above intended for general health purpose, including vitamins, nutrients, steroids, and the like.

(4) Human and Animal Growth Promoting Structural, or Cosmetic Products

Preferred human and animal growth promoting, structural or cosmetic products as defined above include the class of apatite derivatives, for example calcium hydroxyapatite which functions as a bone or dental component, silicon which functions as a tissue modelling component, and analogues, precursors or functional derivatives thereof, bioactive species such as collagen, bioglasses and bioceramics, and components adapted for incorporation as implants into meniscus, cartilage, tissue and the like or for use in sutures or the like and preferably promote growth, modelling, enhancing or reinforcing of collagen, fibroblasts and other natural components of these host structures.

Organic or inorganic components as hereinbefore defined may be selected from tricalcium phosphate or the class of apatite derivatives, for example calcium hydroxyapatite which functions as a bone or dental component and promotes biocompatability, silicon which functions as a tissue modelling component, and analogues, precursors or functioning derivatives thereof, bioactive species such as collagen, bioglasses and bioceramics, other minerals, hyaluran, polyethyleneoxide, CMC (carboxymethylcellulose), proteins, organic polymers, and the like and components adapted for incorporation as implants into meniscus, cartilage, tissue and the like and preferably promote growth, modelling, enhancing or reinforcing of collagen, fibroblasts and other natural components of these host structures.

Function enhancing components as hereinbefore defined may be selected from growth promoters, biocompatibilisers, vitamins, proteins, glycoproteins, enzymes, nucleic acid, carbohydrates, minerals, nutrients, steroids, ceramics and the like, and materials described above as drugs, taking the form of any of these, such as antibiotics (anti bacterial drugs), anti-psychotic drugs and the like. In particular growth factors such as basic Fibroblastic Growth Factor, acid Fibroblastic Growth Factor, Epidermal Growth Factor, Human Growth Factor, Insulin Like Growth Factor, Platelet Derived Growth Factor, Nerve Growth Factor, Vascular Endothelial Growth Factor, Bone Morphogenetic Protein-2, and Transforming Growth Factor

(5) Absorbent biofunctional materials for poisons, toxins and the like may be defined as any natural or synthetic products capable of immobilising by absorption, interaction, reaction or otherwise of naturally occurring or artificially introduced poisons or toxins.

(6) Functioning matter as hereinbefore defined may be selected from any subcellular, cellular or multicellular matter and aggregates and mixtures thereof. Preferably functioning matter is selected from mammalian, plant and bacterial cells including (subcellular) organelles and aggregates thereof including pancreatic islet or liver spheroids and the like, spores, viruses, bacteria and the like;

non cellular matter such as liposomes optionally as carrier of matter such as protein or enzymes which become sensitive to dense phase fluid in presence of liposomic water. Cellular matter is more preferably selected from mammalian and plant prokaryotic and eukaryotic cells and mixtures and aggregates thereof, most preferably mammalian cells selected from fibroblasts, fibrochondrocytes, chondrocytes, bone forming cells such as osteoblasts and osteoclasts, bone marrow cells, hepatocytes, cardiomycytes, blood vessel forming cells, neurons, myoblasts, macrophages, microvascular endothelium cells and mixtures thereof and collagen. Biological functioning matter may be naturally occurring or synthetic, for example cells may be genetically modified or mutated in known manner to incorporate, delete or modify components.

Preferably functioning matter is selected from a component, or precursor, derivative or analogue thereof, of a host structure into which implantation or incorporation is desired and preferably comprises matter intended for growth or repair, shielding, protection, modification or modelling of a human, animal, plant or other living host structure for example the skeleton, organs, dental structure and the like; to combat antagonists; for metabolism of poisons, toxins, waste and the like or for synthesis of useful products by natural processes, for bioremediation, biosynthesis, biocatalysis or the like.

In a particular advantage the process of the invention enables instant production of functioning matter laden extrudates, for example cell laden extrudates, in one step, in contrast with current practice of forming a scaffold and seeding with cells over a 24 or 48 hour period. This may have particular advantages in the delivery for example of stem and progenitor cells to patients.

In a further advantage we believe that the processing may confer a degree of sterilisation by the plasticising fluid, whereby it selectively inactivates non preserved matter, such as bacteria present in the atmosphere and the like.

(7) Organic or inorganic materials for use in dyeing, constructing textiles, electronic materials and the like

(8) SMART materials such as sensors or probes including materials which respond to or otherwise identify substrates which are sought to be detected, for example in environmental applications for contaminant or other detection, or in process control applications for determining components in industrial streams; or materials which respond to stimuli or other influences to change property such as colour changing materials which respond to light or heat and may be used in textiles and the like. Other SMART materials and their applications are well known in the art.

(9) Formulating agents which may be present together with guest matter as hereinbefore defined may be selected from excipients, carriers, supports, binders, diluents, fillers, quick release agents, adhesion promoters, stabilisers, antioxidants, initiators, accelerators, buffers, hardeners, and the like. For example sugars such as glucose derivatives (mannitol or sorbitol) or inorganic salts such as zinc acetate may be present to aid release and maintain activity of active guest matter, for example as a buffer.

The guest matter may be in any desired form suited for the function to be performed, for example in solid form, semi-solid form such as thixotrope or gel form, semi-fluid form or fluid form such as paste or liquid form, and may be miscible or immiscible, soluble or insoluble in the polymer and plasticising fluid. It may be convenient to adapt the guest matter form to render it in preferred form for processing and the function to be performed. The guest matter is preferably in the form of solid particles having particle size selected according to the desired application. Preferably particle size is of similar or of lesser order to that of the composition form, and optionally of any pores, preferably 10⁻⁹ m-10⁻² m, for example of the order of nanometers, micrometers, millimetres or centimetres. Prolonged release of guest matter may be obtained with use of relatively larger extrudates, compared with rapid release obtained with relatively smaller extrudates, for example.

Guest matter may be present in any desired effective amount with respect to polymer. Typical values are therefore 1×10⁻¹² wt % or 1×10⁻⁹ wt % to 99.9 wt %, preferably 1×10⁻¹² to 1×10⁻⁶ or 1×10⁻⁶ to 1 wt %, more preferably 1×10⁻¹² to 1×10⁻⁹, 1×10⁻⁹ to 1×10⁻⁶ or 0.01 or 0.1 to 1 wt % or greater than 0.5 wt % or 1.0 wt % up to 50 wt %. In a particularly preferred embodiment the process is conducted with incorporation of guest matter in low volumes of the order of picogram and nanogram levels with respect to 5 g amounts of polymer. For example, presented as concentration of guest matter on polymer, low volumes in the range 1×10¹ to 1×10³ ng/mg may be present, for example 5 to 150 ng/mg. This is beneficial for most biologically active molecules such as enzymes or protein molecules because their therapeutic concentrations are very low. For example: the therapeutic amount of the growth factor HGF (hepatocyte growth factor) required to provide a therapeutic response in liver cells during liver regeneration process in tissue engineering is 15 ng/ml/day. Proliferative functioning matter may be provided in the process at a desired starting concentration allowing for survival and post processing growth.

For example an extrudate may comprise 80 wt % hydroxyapatite, 10 wt % cells, less than 1 wt % growth factor and more than 1 wt % antibiotic.

Plasticising fluid is selected from carbon dioxide, di-nitrogen oxide, carbon disulphide, aliphatic C₂-₁₀ hydrocarbons such as ethane, propane, butane, pentane, hexane, ethylene, and halogenated derivatives thereof such as for example carbon tetrafluoride or chloride and carbon monochloride trifluoride, and fluoroform or chloroform, C₆-₁₀ aromatics such as benzene, toluene and xylene, C₁-₃ alcohols such as methanol and ethanol, sulphur halides such as sulphur hexafluoride, ammonia, xenon, krypton and the like. Typically these fluids may be brought into supercritical plasticising, preferably conditions at temperature of between 0-300° C. and pressures of 7-1000 bar, preferably 12-800 bar. It will be appreciated that the choice of fluid may be made according to its properties, for example diffusion and as solvent. Preferably the fluid acts as solvent for residual components of polymer substrate but not for guest matter as hereinbefore defined. Choice of fluid may also be made with regard to critical conditions which facilitate the commercial preparation of the polymer as hereinbefore defined.

TABLE 1

| Fluid | Critical Temperature/° C. | Critical Pressure/bar |
|---|---|---|
| Carbon dioxide | 31.1 | 73.8 |
| Ethane | 32.4 | 48.1 |
| Ethylene | 9.3 | 49.7 |
| Nitrous oxide | 36.6 | 71.4 |
| Xenon | 16.7 | 57.6 |
| Fluoroform CHF₃ | 26.3 | 48.0 |
| Monofluoromethane | 42 | 55.3 |
| Tetrafluoroethane | 55 | 40.6 |
| Sulphur hexafluoride | 45.7 | 37.1 |
| Chlorofluoromethane | 29 | 38.2 |
| Chlorotrifluoromethane | 28.9 | 38.7 |
| Nitrogen | −147 | 33.9 |
| Ammonia | 132.5 | 111.3 |
| Cyclohexane | 280.3 | 40.2 |
| Benzene | 289.0 | 48.3 |
| Toluene | 318.6 | 40.6 |
| Trichlorofluoromethane | 198.1 | 43.5 |
| Propane | 96.7 | 41.9 |
| Propylene | 91.9 | 45.6 |
| Isopropanol | 235.2 | 47.0 |
| p-xylene | 343.1 | 34.7 |

Preferably the fluid comprises carbon dioxide optionally in admixture with any further fluids as hereinbefore defined or mixed with conventional solvents, so-called “modifiers”. CO₂ is generally approved by regulatory bodies for medical applications, is chemically inert, leaves no residue and is freely available.

Additional components which may be incorporated during the manufacture of the polymer extrudates, for example initiators, accelerators, hardeners, stabilisers, antioxidants, adhesion promoters, fillers and the like may be incorporated within the polymer or excipient. Markers and tags and the like may be incorporated to trace or detect administration or consumption of the extrudates according to known techniques.

If it is desired to introduce an adhesion promoter into the polymer extrudate, the promoter may be used to impregnate or coat particles of guest matter prior to incorporation with the polymer by means of simple mixing, spraying or other known coating steps, in the presence or absence of fluid as hereinbefore defined. Preferably coating is performed in conjunction with mixing with fluid as hereinbefore defined whereby excellent coating is obtained. For example the adhesion promoter is dissolved in fluid as hereinbefore defined and the solution is contacted with polymer and guest matter particles as hereinbefore defined. Alternatively the adhesion promoter is introduced during the processing whereby it attaches to the guest matter particles in desired manner.

Preferably the total amount of fillers including the guest matter lies in the region of 0.01-99.9 wt %, preferably 0.1-99 wt %; more preferably in excess of 50 or 60 wt %, up to for example 70 or 80 wt %.

The guest matter may be treated prior to or during the incorporation in the polymer with any suitable materials adapted to enhance the performance or mechanical properties thereof. The guest matter may be treated with components such as binders adapted to promote adhesion of matter to the polymeric substrate, dispersants to increase dispersion throughout the substrate and prevent aggregate formation, to increase dispersion as a suspension throughout a plasticising fluid, activators to accelerate any biofunctional effect in situ and the like. Preferably a biofunctional material comprising hydroxapatite may be treated with binding species such as silanes and the like to facilitate increased adhesion of particles to the polymeric substrate.

Without being limited to this theory it is thought that the adhesion promoter attaches to the guest matter thereby exposing or otherwise selecting a binding site which may bind to the polymer.

Preferably the adhesion promoter is soluble in plasticising fluid as hereinbefore defined whereby residual promoter which is not bound to the guest matter or to the polymer is removed by extraction from the product polymer extrudate by the fluid, or the vented gas.

In a further aspect of the invention there is provided a polymer extrudate comprising polymer matrix and guest matter as hereinbefore defined as a solid admixture in extrudate form. Extrudate may be porous or non-porous and may be of varied morphology and porosity as hereinbefore defined. Extrudates are suitably in the form of sheets, films, tubes, cylinders, ribbons, fibrils, fibroids, fibres, woven or non-woven extrudates. Extrudates may be of any suitable dimensions and are preferably of diameter or height and/or width in the range 0.001 to 10 millimetres, more preferably 0.001 millimetres to 2 millimetres, more preferably 0.005 millimetres to 2 millimetres, more preferably 0.005 millimetres to 1 millimetre for example 0.01 to 2 millimetres or 0.05 to 1 millimetre. Extrudates may be of length in excess of 0.01 millimetres, and have no effective upper limit, ie may be continuously produced and collected on a reel or the like, allowing extrudate of km length. Preferably extrudate is of length in the range 0.01 millimetres to 100 metres, preferably 0.05 millimetres to 2 metres more preferably 0.1 to 50 millimetres.

It is a particular feature of the invention that properties of polymer density and porosity and biodegradability may be employed to beneficial effect in release of guest matter, such as drugs and the like in/or in association with the human or animal body or living matter, and/or as structural implants in or in association with the human or animal body or living matter, to be compatible in terms of structural properties of the locus of implantation. Accordingly extrudate may be porous or non-porous.

Moreover extrudate porosity may be selected for a desired mechanical strength and flexibility. In a particular advantage the polymer is adapted to mimic the structure of porous human and animal host structures such as bone, meniscus and cartilage, dental and tissue structures thereby enhancing its suitability as structural or release implant and simultaneously improving biocompatibility thereof.

Porous extrudate may be of closed cell or open cell porosity and may comprise interconnects or the like as known in the polymer art.

We have found that decreasing porosity increases strength and flexibility, whereas more porous extrudates may be of lower strength and brittle. Porosity may be present in one or more orders or magnitude, and may be suitable for either conferring desired mechanical properties or desired guest matter release properties of both. Suitable pores are of the order of macro, meso or micropores as known in the art, in the ranges >50 nm, 2-50 nm and <2 nm respectively. Pore size suitable depends on the intended application, for example for mimic porous host structures as hereinbefore defined or for desired release properties as hereinbefore defined. Pore type and size may be controlled as known in the art by techniques such as rate of release of pressure, for example rapid release causes open pore formation and slow release causes closed pore formation.

Guest matter may be uniformly or non uniformly distributed throughout the extrudate as desired. Guest matter may be present in crystalline form of as a solid dispersion. The extrudate may comprise a combination of guest matter of different types as hereinbefore defined.

In a particular advantage of the invention extrudates are of excellent quality in terms of morphology, porosity and uniformity of incorporated guest matter.

In a further aspect of the invention there is provided a composition comprising polymer extrudate as hereinbefore defined, as a collection of extrudates together with a suitable support, binder, diluent or the like, or which comprising individual extrudate for example as individual scaffolds and the like. Preferably a composition comprises a polymer extrudate in a form selected from cream, gel, syrup, paste, spray, solution, suspension, or shaped body for administration by topical, oral, rectal, parenteral, epicutaneous, subcutaneous, mucosal, intravenous, intramuscular or intrarespiratory application route; as a structure comprising natural metal, plastic, carbon or glass fibre mesh, scrim or rod reinforcing; in a formulation selected from pellets, granules, fillers or cements for bone or teeth inserts or as solid aggregate or monolith pins or crowns as orthopaedic or dental implants; unsupported extrudates of the polymer matrix, as a barrier film, layer, clothing or sheet adapted to enclose or otherwise surround the body or matter to be protected; and combinations thereof.

Preferably a composition comprises extrudate shaped to form a shaped body such as a capsule pellet, tablet, suppository, pessary, colloidal matrix, monolith bolus or the like and which is of shaped size from submicron powders to monoliths of the order of centimeters.

In a further aspect of the invention there is provided an apparatus for use in the preparation of polymer extrudate as hereinbefore defined comprising a pressure chamber adapted for temperature and pressure elevation which may comprise means for mixing the contents, and wherein the pressure chamber includes means for extruding contents via an orifice as hereinbefore defined into a second collection zone at lower pressure. The apparatus comprises means for introduction of reactants and components whilst the pressure chamber is pressurised, as commonly known in the art, and for maintaining a desired pressure during extrusion. Suitably an extrusion orifice comprises a die of desired shape, dimensions and length as hereinbefore defined.

Preferably a pressure chamber is an autoclave which may comprise means for advancing contents from an inlet end or a first chamber region or zone, via a plasticising and optional mixing region or zone to an extrusion region or zone. Advancing means may comprise a screw or piston as known in the art or any suitable equivalent.

In a further aspect of the invention there is provided the use of the extrudate or a composition thereof or the process as hereinbefore defined as a controlled release device such as a device for delivery of a human or animal medical product such as a drug or a medical agent such as an imaging or diagnostic agent as hereinbefore defined; in Pharmaceutical or Veterinary applications for example as a human or animal health or growth promoting structural or cosmetic product, natural or artificial implant, drug delivery or DNA delivery device, tissue engineering device or aid such as such as sutures, and the like; as an anti-microbial for example having bacteria—static or -cidal activity; as a natural or synthetic barrier capable of immobilising e.g. naturally occurring or artificially introduced poisons or toxins by e.g. absorption, interaction or reaction; in Agrochemical or crop protection applications; in the processing of thermally labile fibres for use in dyeing, textiles, electronics etc below the polymer Tg, Tm or melt viscosity, in incorporation of dyes and other thermally labile materials into polymers that cannot be formed by traditional processes e.g. melt extrusion and the like; or in incorporation of surfactants into fibres to control polymer properties.

Preferably a composition as hereinbefore defined is suitable for use as hereinbefore defined, as a pharmacologically active product, preferably a pharmaceutical or veterinary product, a human or animal health or growth promoting, structural or cosmetic product, an agrochemical or crop protection product, a natural or synthetic barrier capable of immobilising naturally occurring or artificially introduced poisons, toxins and the like by absorption, interaction, reaction and the like.

In the case that the extrudate or composition is to be introduced internally to a desired locus, it may be introduced by any desired means such as injection, insertion, ingestion, or the like.

Suitably dry or wet insertion into a human or animal host structure is by any known technique, for example for bone, implanting in orthopaedic and prosthetic applications, implanting as cement or crown in dental applications or dental restructuring, or implanting into a host structure as a slow release implant. The use of the polymer may be for cosmetic/aesthetic or for medical application. It is a particular advantage that a polymer as hereinbefore defined comprising biofunctional material may be inserted in known manner to encourage growth within the host structure whereby the insert becomes integral with the host structure.

Suitably use for release of as hereinbefore defined is by introducing the composition into a desired locus. A non-biodegradable polymer composition may provide release of guest matter by delayed water penetration, restricted rates of substrate diffusion through voids in the polymer matrix and the like, with excretion or surgical removal of matrix from the human or animal body, or removal from any locus as desired. A biodegradable extrudate may provide release in the course of biodegradation, by progressively exposing extrudate to the locus with progressive degradation.

In a further aspect of the invention there is provided a process for preparing polymer extrudate comprising contacting a polymer substrate with a plasticising fluid under dense phase, sub critical or supercritical plasticising conditions of elevated temperature and/or pressure to plasticise the polymer substrate and extruding polymer substrate under dense phase, sub critical or supercritical conditions via an extrusion orifice into a collection zone or a mould with simultaneous or subsequent release of pressure, whereby extrudate is obtained in form conferred by the orifice or the mould characterised in that the process is conducted at temperature of less than or equal to 200° C. Preferably the polymer substrate comprises a thermally labile polymer.

The invention is now illustrated in non limiting manner with reference to the following examples and Figures wherein

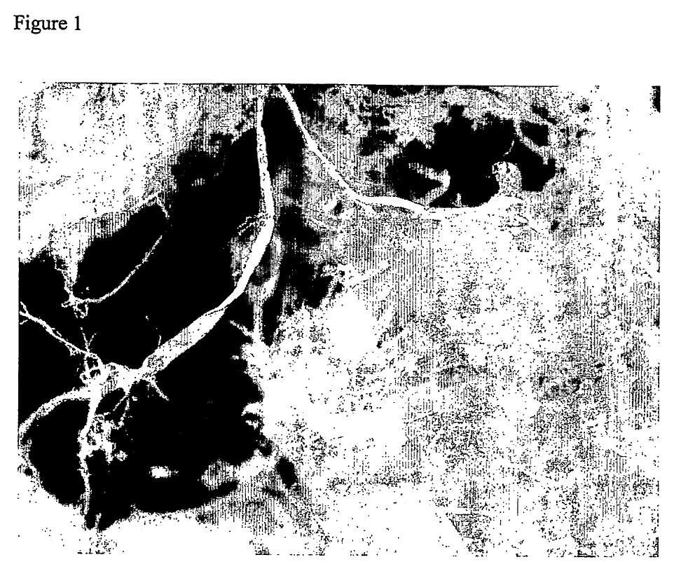

FIGS. 1-3 show images of fibres produced according to the invention.

### COMPARATIVE EXAMPLE

### Powder Formation

Poly(D,L-lactic acid) (MW 8,000) is added to a high pressure autoclave. The autoclave is charged with carbon dioxide at a defined temperature and pressure (ca. 35° C., 300 bar) sufficient to ensure plasticization of polymer by carbon dioxide. Discharging of the contents into a second autoclave at atmospheric pressure, through a short angled nozzle, yields a powder product.

### EXAMPLES OF THE INVENTION

### Example 1

Poly(D,L-lactic acid) (MW 107,000) is added to a high pressure autoclave. The autoclave is charged with carbon dioxide at a defined temperature and pressure (ca. 35° C., 300 bar) sufficient to ensure plasticization of polymer by carbon dioxide. Discharging of the contents into a second autoclave at atmospheric pressure, through a short angled nozzle, yields a solid fibrous-mesh product. The fibrous product is shown in FIG. 1.

### Example 2

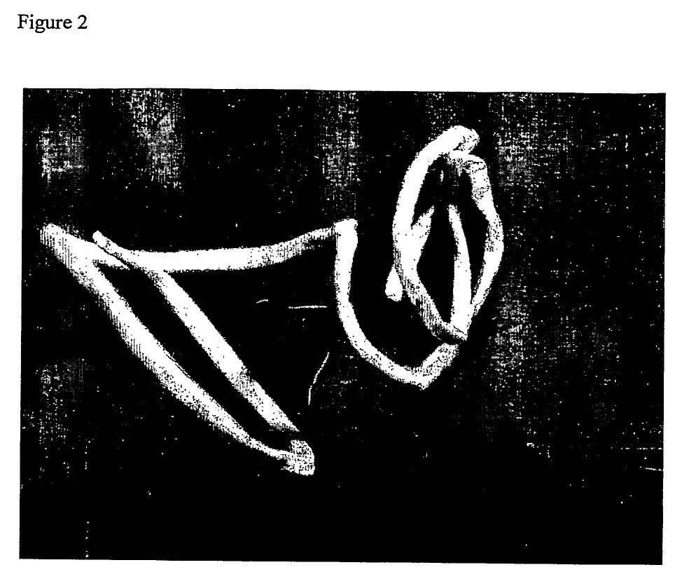

Poly(D,L-lactic acid) (MW 107,000) is added to a high pressure autoclave. The autoclave is charged with carbon dioxide at a defined temperature and pressure (ca. 35° C., 125 bar) sufficient to ensure plasticization of polymer by carbon dioxide. Discharging of the contents into a second autoclave with a back pressure of gas (ca. 90 bar), through a short angled nozzle, yields a more porous single fibrous product. The fibrous product is shown in FIG. 2.

### Example 3

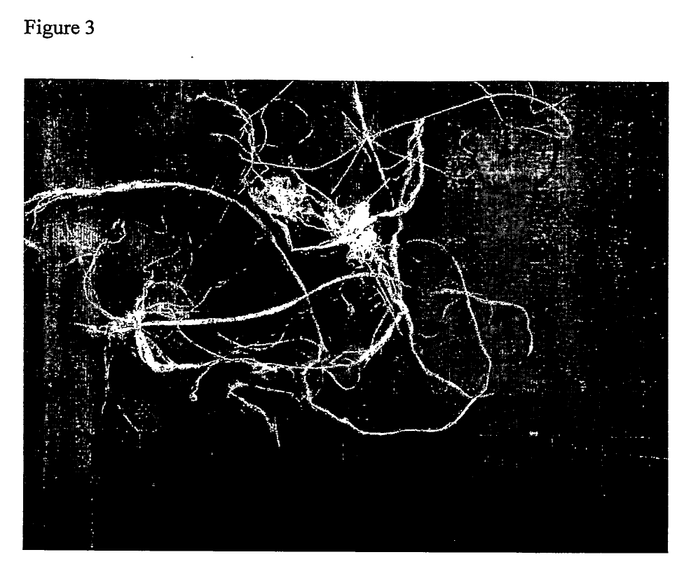

Poly(D,L-lactic acid) (MW 107,000) is added to a high pressure autoclave. The autoclave is charged with carbon dioxide at a defined temperature and pressure (ca. 35° C., 300 bar) sufficient to ensure plasticization of polymer by carbon dioxide. Discharging of the contents into a second autoclave at atmospheric pressure, through a longer angled nozzle, yields a solid fibrous-mesh product of different morphology to Example 1. The fibrous product is shown in FIG. 3.

### Example 4

Poly(glycolic-co-D,L-lactic acid) (MW 158,000) is added to a high pressure autoclave. The autoclave is charged with carbon dioxide at a defined temperature and pressure (ca. 35° C., 300 bar) sufficient to ensure plasticization of polymer by carbon dioxide. Discharging of the contents into a second autoclave at atmospheric pressure, through a long angled nozzle, yields a single solid fibrous product.

### Example 5

Poly(D,L-lactic acid) (MW 71,000) and ribonuclease enzyme powder are added to a high pressure autoclave at a defined ratio (ca. 20:1 weight for weight). The autoclave is charged with carbon dioxide at a defined temperature and pressure (ca. 35° C., 300 bar) sufficient to ensure plasticization of the polymer by carbon dioxide and the contents are mixed together. Discharging of the contents into a second autoclave at atmospheric pressure, through a longer angled nozzle, yields a solid fibrous-mesh product, containing ribonuclease, of similar morphology to Example 1. Upon liberation from the polymer the biological activity of ribonuclease is unaffected by the carbon dioxide processing compared to ribonuclease that has not undergone processing. Standard activity assays described in the literature are used to determine the ribonuclease activity.

### Example 6

Poly(D,L-lactic acid) (MW 71,000) and lysozyme enzyme powder are added to a high pressure autoclave at a defined ratio (ca. 20:1 weight for weight). The autoclave is charged with carbon dioxide at a defined temperature and pressure (ca. 35° C., 300 bar) sufficient to ensure plasticization of the polymer by carbon dioxide and the contents are mixed together. Discharging of the contents into a second autoclave at atmospheric pressure, through a longer angled nozzle, yields a solid fibrous-mesh product, containing lysozyme, of similar morphology to Example 1. Upon liberation from the polymer the biological activity of lysozyme is unaffected by the carbon dioxide processing compared to lysozyme that has not undergone processing. Standard activity assays described in the literature are used to determine the lysozyme activity.

A short angled nozzle as used in the examples is of diameter less than 1 mm and length less than 2 mm and spray angle of 8 to 20°. A long angled nozzle is of diameter less than 1 mm and length from 3 to 8 mm and spray angle of 50 to 75°.

Further aspects and advantages of the invention will be apparent from the foregoing.

1. Process for preparing active polymer extrudate comprising polymer matrix and guest matter, the process comprising contacting a polymer substrate and guest matter with a supercritical fluid under supercritical conditions of elevated temperature and/or pressure to plasticise the polymer substrate and incorporate guest matter and extruding polymer substrate incorporating guest matter under supercritical conditions via an extrusion orifice into a collection zone or a mould with simultaneous or subsequent release of pressure, whereby extrudate is obtained comprising a solid admixture of polymer matrix and guest matter in form conferred by the orifice or the mould wherein extrudate is in the form of tubes, cylinders, rods, ribbons, fibrils, filaments, fibroids or fibres.

2. Process for preparing active polymer extrudate comprising polymer matrix and guest matter, the process comprising contacting a polymer substrate and guest matter with a supercritical fluid under supereritical conditions of elevated temperature and/or pressure to plasticise the polymer substrate and incorporate guest matter and extruding polymer substrate incorporating guest matter under supercritical conditions via an extrusion orifice into a collection zone or a mould with simultaneous or subsequent release of pressure, whereby extrudate is obtained comprising a solid admixture of polymer matrix and guest matter in form conferred by the orifice or the mould wherein extrudate is in the form of sheets or films.

3. Process as claimed in claim 1 or 2 for preparing extrudate suitable for topical, rectal, parenteral, mucosal, epicutaneous, subcutaneous, intravenous or intrarespiratory application routc or as a structural implant in the human or animal body or in living matter.

4. Process according to any of claims 1 to 3 conducted in the substantial absence of additional solvent.

5. Process according to any of claims 1 to 4 conducted at temperature in the range 30° C. to 55° C. and less than or equal to 140° C.

6. Process for preparing active polymer extrudate comprising polymer matrix and guest matter, the process comprising contacting a polymer substrate and guest matter with a supercritical fluid under supercritical conditions of elevated temperature and/or pressure to plasticise the polymer substrate and incorporate guest matter and extruding polymer substrate incorporating guest matter under supercritical conditions via an extrusion orifice into a collection zone or a mould with simultaneous or subsequent release of pressure, whereby extrudate is obtained comprising a solid admixture of polymer matrix and guest matter in form conferred by the orifice or the mould in the form of tubes cylinders, rods, ribbons, fibrils, filaments, fibroids or fibres characterised in that the process is conducted at temperature 30° C. to 55° C. and less than or equal to 140° C. and less tand the Tg, Tm or non-viscous state of the polymer substrate.

7. Process according to any of claims 1 to 6 conducted with polymer substrate of molecular weight in the range 20 to 50 kDa or 50 to 200 kDa.

8. Process as claimed in any of claims 1 to 7 wherein two or more polymer types are contacted with supercritical fluid as discrete components and co-extruded to form a composite extrudate having two or more polymer layers or zones.

9. Process as claimed in any of claims 1 to 8 comprising plural guest entities comprising guest matter of one type for one intended function together with guest matter of another type for a same or different intended function, for example one or more drugs and one or more excipients.

10. Process according to any of claims 1 to 9 conducted with orifice dimensions in the range 0.001-10 millimetre, preferably 0.001-2 millimetre and length in the range 0.1 millimetre to 1 mctre.

11. Process according to any of claims 1 to 10 conducted with orifice of increasing dimension along its length, preferably increasing at a first angle with respect to the axis and optionally at a second angle in respect to the axis at the orifice outlet.

12. Process as claimed in any of claims 1 to 11 wherein an orifice is one of a plurality of orifices which may be independent or which may be adjacently or coaxially or concentrically aligned to form a plurality of simple extrudates or to form a composite extrudate as hereinbefore defined, and nmay additionally or alternatively comprise a solid core or the like, whereby hollow extrudate is obtained for example an annular orifice may provide tubes or cylinders.

13. Process according to any of claims 1 to 12 wherein extrusion is into a collection zone at positive, ambient or negative pressure, which may be greater or less than the supercritical pressure and is preferably in the range 50 to 140 bar or in the range 1 to 50 bar.

14. Process according to any of claims 1 to 13 wherein polymer substrate is selected from any amorphous, semi-crystalline or crystalline polymer, suitably polymers such as polyesters, poly (ortho esters), polyanbydrides, poly(amino acids), poly(pseudo amino acids), polyphosphazencs, azo polymers; vinyl polymers poly(aciylic acid), poly(methacrylic acid), polyacrylamides, polymethacrylamides, polyacrylates, Poly(ethylene glycol), Poly(dimethyl siloxane), Polyurethanes, epoxy, bis-maleimides, methacrylates such as methyl or glycidyl methacrylate, Polycarbonates, Polystyrene and derivatives; carbohydrates, polypeptides and proteins; and copolymers thereof.

15. Process according to any of claims 1 to 14 wherein guest matter is selected from biofunctional or non-biofunctional material including but not limited to:
(1) (pharmaceutical) drugs and veterinary products;
(2) agrochernicals as pest and plant growth control agents;
(3) human and animal healthcare products;
(4) human and animal growth promoting, structural, or cosmetic products including products intended for growth or repair or modelling of the skeleton, organs, dental structure and the like;
(5) absorbent biofunctional materials for poisons, toxins and the like;
(6) functioning matter such as any nutrient dependent, biological matter which is characterised by replication, division, regeneration, growth, proliferation or the like;
(7) organic or inorganic materials for use in dyeing, constructing textiles, electronic materials and the like;
(8) SMART materials.
(9) formulating agents which stabilise or enhance the functional material.

16. Process as claimed in any of claims 1 to 15 wherein guest matter is present in an amount of 1×10⁻¹² to 1×10⁻⁶ or 1×10⁻⁶ to 1 wt %, more preferably in low volumes in the range 1×10⁻¹² to 1×10⁻⁹, 1×10⁻⁹ to 1×10⁻⁶ or 0.01 or 0.1 to 1 wt %.

17. Process as claimed in any of claims 1 to 15 wherein guest matter is present in an amount of 1.0 wt % up to 50 wt %.

18. Polymer extrudate comprising polymer matrix and guest matter as hereinbefore defined in any of claims 1 to 23 as a solid admixture in extrudate form in the form of tubes, cylinders, rods, ribbons, fibrils, filaments, fibroids or fibres, wherein the polymer matrix comprises polymer of molecular weight in the range 20 to 50 kDa or 50 to 200 kDa.

19. Polymer extrudate as claimed in claim 18 suitable for topical, rectal, parenteral, mucosal, epicutaneous, subcutaneous, intravenous or intrarespiratory application route or as a structural implant in the human or animal body or in living matter wherein guest matter is present in an amount of 1×10⁻¹² to 1×10⁻⁶ or 1×10⁻⁶ to 1 wt %, more preferably in low volumes in the range 1×10⁻¹² to 1×10⁻⁹, 1×10⁻⁹ to 1×10⁻⁶ or 0.01 or 0.1 to 1 wt %.

20. Polymer extrudate as claimed in claim 18 suitable for topical, rectal, parenteral, mucosal, epicutaneous, subcutaneous, intravenous or intrarespiratory application route or as a structural implant in the human or animal body or in living matter wherein guest matter is present in an amount of 1.0 wt % up to 50 wt %.

21. Apparatus for use in the preparation of polymer extrudate using the process as hereinbefore defined in any of claims 1 to 17 comprising a pressure vessel adapted for temperature and pressure elevation which may comprise means for mixing the contents, and wherein the pressure vessel includes means for extruding contents via an orifice as hereinbefore defmcd into a second collection vessel at lower pressure.

22. Extrudate as claimed in any of claims 18 to 20 or a composition thereof or a product of the process as claimed in any of claims 1 to 17 for use as a controlled release device such as a drug delivery device; in Pharmaceutical or Veterinary applications for example as a human or animal health or growth promoting structural or cosmetic product, natural or artificial implant, drug delivery or DNA delivery device; as an anti-microbial for example having bacteria-static or -cidal activity; as a natural or synthetic barrier capable of immobilising e.g. naturally occurring or artificially introduced poisons or toxins by e.g. absorption, interaction or reaction; in Agrochernical or crop protection applications; in the processing of thermally labile fibres for use in dying, textiles, electronics etc below the polymer Tg, Tm or melt viscosity- in incorporation of dyes and other thennally labile materials into polymers that cannot be formed by traditional processes e.g. melt extrusion and the like; or in incorporation of surfactants into fibres to control polymer properties.

23. Process for preparing polymer extrudate comprising contacting a polymer substrate with a supercritical fluid under supercritical conditions of elevated temperature and/or pressure to plasticise the polymer substrate and extruding polymer substrate under supercritical conditions via an extrusion orifice into a collection zone or a mould with simultaneous or subsequent release of pressure, whereby extrudate is obtained in form conferred by the orifice or the mould in the form of tubes, cylinders, rods, ribbons, fibrils, filaments, fibroids or fibres characterised in that the process is conducted at temperature of 30° C. to 55° C. and less than or equal to 140° C.

24. Process for preparing polymer extrudate comprising contacting a polymer substrate with a supercritical fluid under supercritical conditions of elevated temperature and/or pressure to plasticise the polymer substrate and extruding polymer substrate under supercrtical conditions via an extrusion orifice into a collection zone or a mould with simultaneous or subsequent release of pressure, whereby extrudate is obtained in form conferred by the orifice or the mould in the form of sheets or films characterised in that the process is conducted at temperature of 30° C. to 55° C. and less than or equal to 140° C.

25. Process as claimed in claim 23 or 24 wherein polymer substrate comprises a thermally labile polymer, for example, poly(acrylonitrile) and copolymers thereof.
